(19) United States
(12) Patent Application Publication
(10) Pub. No.: US 2024/0325002 A1
(43) Pub. Date: Oct. 3, 2024

(54) ACTIVE BIOPSY APPARATUS AND SYSTEM WITH NEEDLE PASSAGE ABLATION FUNCTION, AND METHOD

(71) Applicant: SHANGHAI MEDVIDA MEDICAL TECHNOLOGY CO. LTD., Shanghai (CN)

(72) Inventor: Shujun TANG, Shanghai (CN)

(73) Assignee: SHANGHAI MEDVIDA MEDICAL TECHNOLOGY CO. LTD., Shanghai (CN)

(21) Appl. No.: 18/704,943

(22) PCT Filed: Nov. 24, 2021
(86) PCT No.: PCT/CN2021/132669

§ 371 Date: Apr. 26, 2024

(30) Foreign Application Priority Data
Oct. 29, 2021 (CN) 202111269656.X

(51) Int. Cl. A61B 10/02; A61B 10/06; A61B 18/00; A61B 18/12; A61B 18/14

(52) U.S. Cl. CPC A61B 10/0233; A61B 10/06; A61B 18/1206; A61B 18/1477; A61B 2018/00577; A61B 2018/00642; A61B 2018/00791; A61B 2018/1425

(57) ABSTRACT

An active biopsy apparatus with a needle passage ablation function. The active biopsy apparatus comprises a radio frequency generator, a biopsy cannula and a biopsy tool, wherein the biopsy cannula is an active device, and is connected to the radio frequency generator by means of a connecting line; and a cannula wall of the biopsy cannula is provided with a radio frequency ablation electrode, and the biopsy tool is movably arranged inside the biopsy cannula. An active biopsy system with a needle passage ablation function, wherein a host system is arranged in a radio frequency generator, and the host system comprises a temperature monitoring module and a radio frequency ablation module. A radio frequency ablation system is compatible with a biopsy tool, such that the passive puncturing biopsy tool becomes part of an active system, and can be used for preventing needle passage bleeding and for needle passage implanting.

### CROSS REFERENCE TO THE RELATED APPLICATIONS

This application is the national phase entry of International Application No. PCT/CN2021/132669, filed on Nov. 24, 2021, which is based upon and claims priority to Chinese Patent Application No. 202111269656.X, filed on Oct. 29, 2021, the entire contents of which are incorporated herein by reference.

### TECHNICAL FIELD

The invention relates to the technical field of medical instruments, and in particular to an active biopsy apparatus and system with a needle passage ablation function, and an implementation method thereof.

### BACKGROUND

Tissue biopsy is the most important technical means for tumor diagnosis. The most common and serious complication of resulting injury is hemorrhage, which may lead to death in severe cases. Currently available biopsy tools on the market, with or without a biopsy cannula, are passive devices and do not currently have a hemostatic function. There are two commonly used tools in the clinic, one is an active radio frequency ablation system and another one is a disposable biopsy tool. The existing radio frequency ablation system is commonly used for tumor ablation, and the commonly used power output and ablation range are large. In addition, the existing tools for performing the biopsy are all passive devices. Since the passive devices do not have the needle passage ablation function, during the needle withdrawal process, it is easy to cause needle passage bleeding and implanting, leading to complications and seriously affecting the rehabilitation of patients.

Chinese utility model patent (Granted publication No. CN210582634U) discloses a portable needle passage ablation system, including a bipolar needle body and a connecting handle. The bipolar needle body is composed of a needle tube component and a needle core component. The needle body is inserted into a bipolar needle interface via a connecting cable. The function of the needle tube component in the system is equivalent to that of a trocar, so that a biopsy cannula is established. The needle core component is inserted into the needle tube component after the biopsy is completed, so as to constitute an ablation motor as a whole. The main machine is powered by a battery and operated by one key. It is portable. The utility model differs from the invention in that the utility model emphasizes a portable function, with the battery power used in the device to perform ablation without external power supply, so that the ablation process is more convenient. The invention solves the problems of needle passage implanting and bleeding caused by the biopsy surgery by improving and adding a low-power ablation function to the conventional biopsy device, and performs low-power radio frequency ablation by adding a temperature measurement device in a way that the biopsy device is designed to be active.

### SUMMARY

In view of the above-described deficiencies of the prior art, the invention combines an active radio frequency ablation system including two parts, a low-power radio frequency host and a radio frequency electrode needle, and a disposable biopsy tool with a channel. The invention provides a biopsy tool with a needle passage ablation for percutaneous, laparoscopic or bronchoscopic access and a system composition, which effectively solves the problems of needle passage bleeding and needle passage implanting of conventional passive biopsy tools. To this end, the invention also provides an implementation method of an active biopsy system with a needle passage ablation function.

In order to solve the above-mentioned technical problems, the technical solution provided by the invention is as follows.

In a first aspect, the invention provides an active biopsy apparatus with a needle passage ablation function, including a 400-500 KHz radio frequency generator, a biopsy cannula (simultaneously having the function of a radio frequency ablation electrode) and a biopsy tool. The biopsy cannula is an active device and is connected to the radio frequency generator by means of a connecting line; and a cannula wall of the biopsy cannula is provided with a radio frequency ablation electrode, and the biopsy tool is movably arranged inside the biopsy cannula.

The above-mentioned active biopsy apparatus with needle passage ablation function further comprises an ablation electrode middle pole, wherein the ablation electrode intermediate pole is an electrode needle; the electrode needle is movably arranged inside the biopsy cannula; an insulation layer is arranged between an inner wall of the electrode needle and an outer wall of the biopsy cannula to electrically isolate the two components; and a tail portion of the electrode needle is connected to a radio frequency port of the radio frequency generator by the connecting line. A temperature measurement probe is provided at a front end of the outer wall of the biopsy cannula. When the ablation function needs to be used, the biopsy tool in the biopsy cannula is taken out, the outer sleeve of the biopsy cannula with a connecting line is connected to a radio frequency output port of the host, and the ablation function is activated by the host, so that needle passage ablation can be achieved.

Alternatively, the above-mentioned active biopsy apparatus with the needle passage ablation function further comprises a negative radio frequency plate (usually attached to the surface of the human body), wherein the radio frequency negative plate is an electrode band; the radio frequency generator comprises two radio frequency ports respectively connected to the biopsy cannula and the electrode band by the connecting line. A temperature measurement probe is provided at a front end of the outer wall of the biopsy cannula, and the ablation temperature is measured in real time after the ablation electrode is turned on and uploaded to the radio frequency generator.

According to the active biopsy apparatus with the needle passage ablation function as described above, the biopsy tool is a disposable biopsy brush or a disposable biopsy forceps.

In a second aspect of the invention, an active biopsy system with a needle passage ablation function is provided, comprising the active biopsy apparatus with the needle passage ablation function as described above, wherein radio frequency generator has a host system arranged therein; and the host system comprises a temperature monitoring module and a radio frequency ablation module. The radio frequency generator has a maximum radio frequency output power of 100 W. After the set upper temperature limit of the radio frequency generator is reached, it is operated according to a predetermined holding time, and the normal ablation holding time is 3-8 s. According to the temperature and time of the system, the system automatically turns off the ablation function.

In a third aspect, the invention provides an implementation method of an active biopsy system with a needle passage ablation function, the method comprising the steps of:
step 1, using a biopsy cannula in combination with a biopsy tool, and placing the same on a target lesion for biopsy sampling, wherein, at this time, the biopsy cannula does not need to be connected with a radio frequency generator;
step 2, withdrawing the biopsy tool from the biopsy cannula to obtain a biopsy sample;
step 3, then connecting the biopsy cannula to the radio frequency generator via a connecting line;
step 4, operating the radio frequency generator to start an ablation mode, starting to work the temperature measurement probe, and feeding back the ablation temperature to the system in real time; and
step 5, withdrawing the needle after the ablation is completed.

According to above-mentioned implementation method of the active biopsy system with the needle passage ablation function, in the step 4, when the temperature measured by the temperature measurement probe exceeds 50° C., the temperature monitoring module in the system feeds back a signal to the radio frequency ablation module which starts to calculate the ablation time. After the ablation time reaches the set value of system, the system automatically stops ablation, and the ablation is ended.

Compared with the prior art, the invention has the following beneficial effects.

The invention solves the problems of needle passage implanting and bleeding caused by the biopsy surgery by improving and adding a low-power ablation function to the conventional biopsy device, and performs low-power radio frequency ablation by adding a temperature measurement device in a way that the biopsy device is designed to be active. This device adopts a low-power radio frequency function and does not adopt a water-cooling system, and belongs to an ablation apparatus only used for the needle passage ablation function. The invention is based on the principles of existing radio frequency ablation systems to improve the design of commonly used biopsy tools so that the improved biopsy tool has the function of performing a biopsy, and is modified to be compatible with the radio frequency ablation system having the function of needle passage ablation. The invention effectively solves the problems of needle passage bleeding and needle passage implanting caused by conventional passive biopsy tools, and reduces the complications of biopsy procedures.

The conception, the specific structure and the technical effects of the invention will be further described with reference to the accompanying drawings, so that the objects, features and effects of the invention can be fully understood.

### BRIEF DESCRIPTION OF THE DRAWINGS

FIG. 1 is a structurally schematic view according to Embodiment 1 of the invention;

FIG. 2 is a structurally schematic view according to Embodiment 2 of the invention; and

FIG. 3 is a schematic view showing a use state according to Embodiment 2 of the invention.

In the drawings, the reference numerals are as follows:
biopsy cannula 1, biopsy tool 2, electrode needle 3, temperature measurement probe 4, connecting line 5, radio frequency generator 6, radio frequency port 61, electrode band 7.

### DETAILED DESCRIPTION OF THE EMBODIMENTS

In order to make it easy to understand the technical means, creation characteristics, achievement of purpose and efficacy of the invention, the invention is further elaborated in combination with specific illustrations. However, the invention is not limited to the following embodiments.

It should be understood that the structure, proportion, size, etc. shown in the attached diagram of this specification are only for the understanding and reading of the person familiar with the technology, and are not for the limitation of the implementation of the invention. Thus, they have no technical significance. Any structural modification and change of proportion relationship or size adjustment, without affecting the effects that the invention can produce and the purposes that can be achieved, all shall still fall within the scope covered by the technical content disclosed by the invention.

### Embodiment 1

As shown in FIG. 1, an active biopsy apparatus with a needle passage ablation function includes a biopsy cannula 1. A temperature measurement probe 4 is arranged on an outer wall of the biopsy cannula 1 close to the front end. The biopsy cannula 1 can be used as a radio frequency ablation electrode by connecting to a system host. An electrode needle 3 can also be inserted as an ablation electrode, and can be detachably arranged in a hollow channel of the biopsy cannula 1. The biopsy cannula 1 is an active device and is connected to the radio frequency generator via a connecting line 5. The electrode needle 3 is connected to the radio frequency generator via the connecting line 5. The biopsy tool is a disposable electrode forceps or a disposable biopsy brush, which is placed in the biopsy cannula 1 for use.

The apparatus is used as follows.

The biopsy cannula 1 serves as one of the poles of the needle passage ablation radio frequency electrode, does not need to be connected to the radio frequency generator 6 at the beginning of the operation, and is used as a passive device in the biopsy operation. It cooperates with the biopsy tool to perform the biopsy. After the biopsy operation is completed, the biopsy tool is withdrawn from the biopsy cannula 1 and a biopsy biopsy sample is properly stored. At this time, the biopsy cannula 1 is kept stationary, the electrode needle 3 is placed in the biopsy cannula 1. The biopsy cannula 1 and the electrode needle 3 are respectively connected to a radio frequency port 61 provided on the radio frequency generator 6 via the connecting line 5. The radio frequency generator 6 is turned on. The operator has a system setup for the radio frequency generator 6. The ablation mode is started with one key to perform needle passage ablation. At this time, the temperature measurement probe 4 on the outer wall of the biopsy cannula 1 starts to work to measure the ablation temperature of the biopsy cannula 1 in real time and upload the temperature data to the system. When the ablation temperature reaches 50° C., the system starts to calculate the ablation time and keep the ablation time for 2-3 s, and the system automatically stops the ablation function. After the ablation is completed, the radio frequency generator 6 is turned off and the needle can be withdrawn.

### Embodiment 2

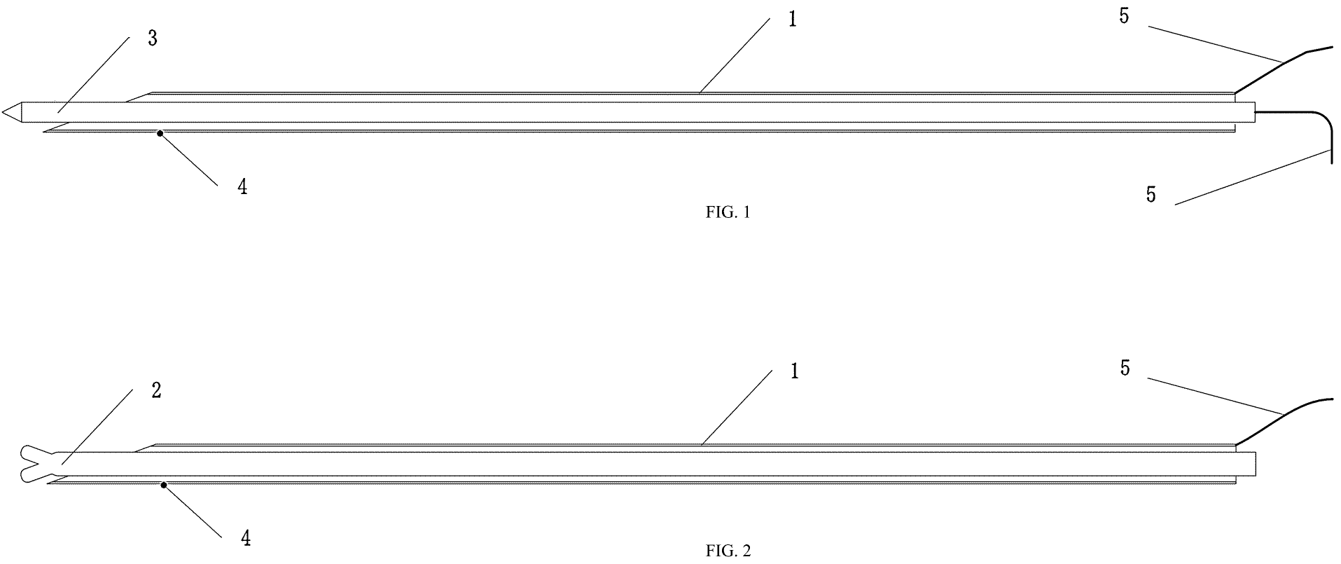

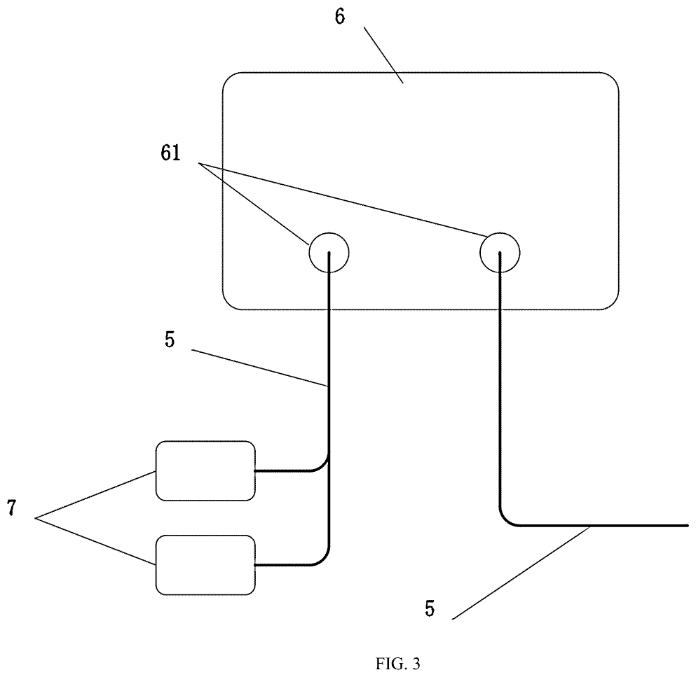

As shown in FIGS. 2 and 3, an active biopsy apparatus with a needle passage ablation function includes a biopsy cannula 1 and a radio frequency generator 6. The cannula wall of the biopsy cannula 1 is connected to an ablation host via a connecting line and works as a radio frequency ablation electrode component. The electrode band 7 is a radio frequency negative plate (usually attached to the surface of the human body), and there are two electrode bands 7, which can be respectively bound to two thighs of a patient during use. The biopsy tool 2 is a disposable biopsy forceps placed in the biopsy cannula 1. The radio frequency generator 6 is provided with two radio frequency ports 61. The electrode band 7 is connected to one radio frequency port 61 via the connecting line 5. The tail portion of the biopsy cannula 1 is connected to the other radio frequency port 61 via the connecting line 5.

The apparatus is used as follows.

The two electrode bands 7 are respectively bound on the two thighs of the patient. The biopsy cannula 1 serves as one pole of the radio frequency electrode for needle passage ablation. The biopsy cannula 1 does not need to be connected to the radio frequency generator 6 in the biopsy step, is only used as a passive device in the biopsy operation, and performs the biopsy operation in cooperation with the biopsy tool 2. After the biopsy operation is completed, the biopsy tool 2 is withdrawn from the biopsy cannula 1 and the biopsy sample is properly saved. At this time, the biopsy cannula 1 is kept stationary, and the electrode band 7 is connected to the radio frequency port 61 of the radio frequency generator 6 via the connecting line 5. The connecting line 5 of the biopsy cannula 1 is connected to the radio frequency port 61 of the radio frequency generator 6. At this time, the ablation operation is performed, and the radio frequency generator 6 is turned on. The operator operates the radio frequency generator 6 to set the system to turn on the ablation mode. At this time, the temperature measurement probe 4 on the outer wall of the biopsy cannula 1 starts to work. The temperature measurement probe 4 is located at the front end of the biopsy cannula 1 and located in the ablation area, and can measure the ablation temperature of the biopsy cannula 1 in real time and upload the temperature data to the system. When the ablation temperature reaches 50° C., the ablation time is calculated in the system. When the set temperature range value is reached, the ablation time is maintained for 3-8 s. The specific maintenance time is determined according to the application. The system automatically stops the ablation function. Since only the needle passage needs to be ablated for hemostasis and implant prevention, the ablation power and ablation time need not be too long. After the ablation is completed, the operator can turn off the radio frequency generator 6 and safely withdraw the needle. At this time, no needle passage implanting will occur during the needle withdrawal.

The invention has developed the radio frequency ablation technology to have a more accurate radio frequency generator output power by system optimization and achieve the ablation by low power output. Since the maximum output power of generator is 100 W, usually not more than 50 W in practical use, this apparatus does not need to be provided with a separate cooling system, and a hollow system design without water cooling is selected. The invention combines the conventional passive puncturing tool with the active ablation electrode technology, adds the local ablation function in the conventional biopsy procedure, and controls the ablation temperature, so as to completely solve the problems of needle passage bleeding and needle passage implanting caused by the puncture needle. The invention improves existing biopsy puncture outfits by adding the needle passage ablation function to the biopsy tool, allowing the biopsy device to perform ablation and hemostasis in addition to the biopsy function. Multiple functions are performed with a single apparatus.

Specific embodiments of the invention are described in detail above. It will be appreciated by persons skilled in the art that numerous modifications and variations can be made to the invention without departing from the scope of the invention. Therefore, the technical solutions which can be obtained by a person skilled in the art through logical analysis, reasoning or limited experimentation on the basis of the prior art according to the idea of the invention are within the scope of protection defined by the claims.

What is claimed is:

1. An active biopsy apparatus with a needle passage ablation function, comprising a radio frequency generator, a biopsy cannula and a biopsy tool, wherein the biopsy cannula is an active device and is connected to the radio frequency generator by a connecting line; and a cannula wall of the biopsy cannula is provided with a radio frequency ablation electrode, and the biopsy tool is movably arranged inside the biopsy cannula;
the active biopsy apparatus further comprises an ablation electrode intermediate pole; the ablation electrode intermediate pole is an electrode needle; the electrode needle is movably arranged inside the biopsy cannula; an insulation layer is arranged between an inner wall of the electrode needle and an outer wall of the biopsy cannula; and a tail portion of the electrode needle is connected to a radio frequency port of the radio frequency generator by the connecting line;
the active biopsy apparatus further comprises a negative radio frequency plate; the negative radio frequency plate is an electrode band; the radio frequency generator comprises two radio frequency ports respectively connected to the biopsy cannula and the electrode band by the connecting line;
wherein in a biopsy step, the biopsy cannula is used only as a passive device, cooperating with the biopsy tool to carry out a biopsy operation; after the biopsy operation is completed, the biopsy cannula is connected to the radio frequency generator through the connecting line, and the biopsy cannula carries out ablation and hemostasis for a needle channel.

2. The active biopsy apparatus according to claim 1, wherein a temperature measurement probe is provided at a front end of the outer wall of the biopsy cannula.

3. The active biopsy apparatus according to claim 1, wherein the biopsy tool is a disposable biopsy brush or a disposable biopsy forceps.

4. An active biopsy system with a needle passage ablation function, comprising the active biopsy apparatus with the needle passage ablation function according to claim 3, wherein a host system is arranged in the radio frequency generator; and the host system comprises a temperature monitoring module and a radio frequency ablation module.

5. The active biopsy system according to claim 4, wherein the radio frequency generator has a maximum radio frequency output power of 100 W.

6. The active biopsy system according to claim 5, wherein the radio frequency generator is set with an ablation time of 3-8 seconds.

7. The active biopsy system according to claim 4, wherein the active biopsy system performs an active biopsy operation with a needle tract ablation, wherein the active biopsy operation with the needle tract ablation performs the following processing:
step 1, using a biopsy cannula in combination with the biopsy tool, and placing the biopsy cannula and the biopsy tool on a target lesion for biopsy sampling, wherein, at this time, the biopsy cannula does not need to be connected with the radio frequency generator;
step 2, withdrawing the biopsy tool from the biopsy cannula to obtain a biopsy sample;
step 3, connecting the biopsy cannula to the radio frequency generator via the connecting line;
step 4, operating the radio frequency generator to start an ablation mode, starting to work a temperature measurement probe, and feeding back an ablation temperature to the active biopsy system in real time; and
step 5, withdrawing a needle after the needle tract ablation is completed.

8. The active biopsy system according to claim 7, wherein in the step 4, when a temperature measured by the temperature measurement probe exceeds 50° C., the temperature monitoring module in the active biopsy system feeds back a signal to the radio frequency ablation module, wherein the radio frequency ablation module starts to calculate an ablation time.

9-10. (canceled)

11. The active biopsy apparatus according to claim 2, wherein the biopsy tool is a disposable biopsy brush or a disposable biopsy forceps.

12. The active biopsy system according to claim 5, wherein the active biopsy system performs an active biopsy operation with a needle tract ablation, wherein the active biopsy operation with the needle tract ablation performs the following processing:
step 1, using the biopsy cannula in combination with the biopsy tool, and placing the biopsy cannula and the biopsy tool on a target lesion for biopsy sampling, wherein, at this time, the biopsy cannula does not need to be connected with the radio frequency generator;
step 2, withdrawing the biopsy tool from the biopsy cannula to obtain a biopsy sample;
step 3, connecting the biopsy cannula to the radio frequency generator via the connecting line;
step 4, operating the radio frequency generator to start an ablation mode, starting to work a temperature measurement probe, and feeding back an ablation temperature to the active biopsy system in real time; and
step 5, withdrawing a needle after the needle tract ablation is completed.

13. The active biopsy system according to claim 6, wherein the active biopsy system performs an active biopsy operation with a needle tract ablation, wherein the active biopsy operation with the needle tract ablation performs the following processing:
step 1, using the biopsy cannula in combination with the biopsy tool, and placing the biopsy cannula and the biopsy tool on a target lesion for biopsy sampling, wherein, at this time, the biopsy cannula does not need to be connected with the radio frequency generator;
step 2, withdrawing the biopsy tool from the biopsy cannula to obtain a biopsy sample;
step 3, connecting the biopsy cannula to the radio frequency generator via the connecting line;
step 4, operating the radio frequency generator to start an ablation mode, starting to work a temperature measurement probe, and feeding back an ablation temperature to the active biopsy system in real time; and
step 5, withdrawing a needle after the needle tract ablation is completed.
